(19) United States
(12) Patent Application Publication
(10) Pub. No.: US 2025/0153161 A1
(43) Pub. Date: May 15, 2025

(54) DISPOSABLE FLUID CASSETTE FOR FLUID MANAGEMENT SYSTEM

(71) Applicant: BOSTON SCIENTIFIC SCIMED, INC., Maple Grove, MN (US)

(72) Inventors: Linda Maher, Tullaroan (IE); Angelo Frank Szychowski, Mendocino, CA (US); John Peter Cannistraro, Boston, MA (US); Bryce Hodsdon, East Boston, MA (US)

(73) Assignee: BOSTON SCIENTIFIC SCIMED, INC., Maple Grove, MN (US)

(21) Appl. No.: 18/941,909

(22) Filed: Nov. 8, 2024

Related U.S. Application Data
(60) Provisional application No. 63/597,551, filed on Nov. 9, 2023.

(51) Int. Cl. B01L 3/00; B01L 7/00

(52) U.S. Cl. CPC B01L 3/502; B01L 7/00; B01L 2200/0684

(57) ABSTRACT

A fluid cassette for a fluid management system, including a housing defining a fluid pathway therethrough, an inflow tubing extending from the housing, and an outflow tubing extending from the housing. The fluid pathway includes a fluid dampening chamber having a fluid inlet configured for fluid ingress into the fluid dampening chamber and a fluid outlet configured for fluid egress from the fluid dampening chamber, where an upper extent of the fluid outlet is lower than an upper extent of the fluid inlet. The fluid pathway also includes a bifurcated fluid pathway including a first branch of the bifurcated fluid pathway and a second branch of the bifurcated fluid pathway, and a mixing fluid channel for mixing heated fluid exiting the bifurcated fluid pathway.

### CROSS REFERENCE TO RELATED APPLICATIONS

This application claims the benefit of and priority to U.S. Provisional Patent Application Ser. No. 63/597,551, filed on Nov. 9, 2023, the disclosure of which is incorporated herein by reference.

### TECHNICAL FIELD

The disclosure is directed to a fluid management system. More particularly, the disclosure is directed to a disposable fluid cassette of a fluid management system.

### BACKGROUND

Flexible ureteroscopy (fURS), gynecology, and other endoscopic procedures require the circulation of fluid for several reasons. Fluid management systems may be used to deliver fluid to an anatomical cite from a reservoir at a desired pressure and/or flow rate via a peristaltic or roller pump. Fluid management systems may adjust the flow rate and/or pressure at which fluid is delivered from the reservoir based on data collected from a procedural device, such as, but not limited to, pressure readings sensed and/or obtained by the fluid management system. The fluid management system may utilize a disposable fluid tubing set installed with a pump console to provide the fluid to the patient. There is an ongoing need to provide alternative configurations of the components of fluid management systems, to facilitate the use thereof.

### BRIEF SUMMARY

This disclosure provides design, material, manufacturing method, and use alternatives for components of a fluid management system.

A first example is a fluid cassette for a fluid management system. The fluid cassette includes a housing defining a fluid pathway therethrough, an inflow tubing extending from the housing, and an outflow tubing extending from the housing. The fluid pathway includes a fluid dampening chamber having a fluid inlet configured for fluid ingress into the fluid dampening chamber and a fluid outlet configured for fluid egress from the fluid dampening chamber. An upper extent of the fluid outlet is lower than an upper extent of the fluid inlet.

Alternatively or additionally to any of the examples above, in another example, the fluid inlet and the fluid outlet are located on opposite sides of the fluid dampening chamber.

Alternatively or additionally to any of the examples above, in another example, the fluid dampening chamber is configured such that a fluid level between a volume of air and a volume of fluid within the fluid dampening chamber is higher than the upper extent of the fluid outlet.

Alternatively or additionally to any of the examples above, in another example, the volume of air is at least 38 milliliters.

Alternatively or additionally to any of the examples above, in another example, the fluid pathway includes a first air vent chamber and an ascending fluid channel interconnecting the fluid dampening chamber and the air vent chamber.

Alternatively or additionally to any of the examples above, in another example, the fluid pathway includes a bifurcated fluid pathway including a first branch of the bifurcated fluid pathway and a second branch of the bifurcated fluid pathway, and a descending fluid channel interconnecting the first air vent chamber with the bifurcated fluid pathway.

Alternatively or additionally to any of the examples above, in another example, the fluid pathway includes a second air vent chamber and a mixing fluid channel interconnecting the bifurcated fluid pathway with the second air vent chamber.

Alternatively or additionally to any of the examples above, in another example, the fluid cassette includes an interior wall separating the first air vent chamber from the second air vent chamber.

Another example is a fluid cassette for a fluid management system. The fluid cassette includes a housing defining a fluid pathway therethrough, an inflow tubing extending from the housing, and an outflow tubing extending from the housing. The fluid pathway includes a first air vent chamber and a second air vent chamber. The first air vent chamber and the second air vent chamber are fluidly connected by a fluid pathway therebetween.

Alternatively or additionally to any of the examples above, in another example, the fluid pathway interconnecting the first air vent chamber and the second air vent chamber is a bifurcated fluid pathway including a first branch of the bifurcated fluid pathway and a second branch of the bifurcated fluid pathway.

Alternatively or additionally to any of the examples above, in another example, the fluid cassette includes a first hydrophobic membrane positioned at an interface between the first air vent chamber and a first air chamber. Air may pass through the first hydrophobic membrane into the first air chamber while fluid in the first air vent chamber is prevented from passing through the first hydrophobic membrane.

Alternatively or additionally to any of the examples above, in another example, the fluid cassette includes a second hydrophobic membrane positioned at an interface between the first air vent chamber and a second air chamber. Air may pass through the second hydrophobic membrane into the second air chamber while fluid in the second air vent chamber is prevented from passing through the second hydrophobic membrane.

Alternatively or additionally to any of the examples above, in another example, wherein the first air chamber is interconnected with the second air chamber.

Alternatively or additionally to any of the examples above, in another example, the first air chamber and the second air chamber are configured to vent air to atmosphere through an air vent valve.

Alternatively or additionally to any of the examples above, in another example, the first air vent chamber includes a deflector configured as an interior wall, wherein an upper extent of the deflector in the first air vent chamber is located closer to an upper extent of the first air vent chamber than to a lower extent of the first air vent chamber.

Alternatively or additionally to any of the examples above, in another example, the second air vent chamber includes a deflector configured as an interior wall, wherein an upper extent of the deflector of the second air vent chamber is located closer to an upper extent of the second air vent chamber than to a lower extent of the second air vent chamber.

Another example is a fluid cassette for a fluid management system. The fluid cassette includes a housing defining a fluid pathway therethrough, an inflow tubing extending from the housing, and an outflow tubing extending from the housing. The fluid pathway includes a bifurcated fluid pathway including a first branch of the bifurcated fluid pathway and a second branch of the bifurcated fluid pathway.

Alternatively or additionally to any of the examples above, in another example, fluid exiting the first and second branches of the bifurcated fluid pathway enters a fluid mixing channel configured to generate a turbulent flow to mix the fluid exiting the first and second branches of the bifurcated fluid pathway.

Alternatively or additionally to any of the examples above, in another example, the first branch of the bifurcated fluid pathway is a first fluid warming pathway and the second branch of the bifurcated fluid pathway is a second fluid warming pathway.

Alternatively or additionally to any of the examples above, in another example, the fluid cassette includes a stack of heating plates arranged in the first and second fluid warming pathways.

Alternatively or additionally to any of the examples above, in another example, the stack of heating plates includes a plurality of spaced apart annular plates configured to permit fluid to pass therebetween.

Alternatively or additionally to any of the examples above, in another example, each of the annular plates includes one or more dimples extending from a flat surface of the annular plate, wherein the one or more dimples of one of the annular plates is configured to contact a second one of the annular plates to space the first annular plate away from the second annular plate.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a perspective view of the front side of the fluid cassette of the disposable tubing set of FIG. 2;

FIG. 4 is perspective view of the rear side of the fluid cassette of the disposable fluid tubing set of FIG. 2;

FIG. 5 is a cross-sectional view of the fluid cassette of FIGS. 3 and 4 showing the internal fluid pathway therethrough;

FIG. 6 is an enlarged cross-sectional view of a portion of the fluid cassette of FIG. 5 showing the dampening chamber;

FIG. 7 is an enlarged cross-sectional view of a portion of the fluid cassette of FIG. 5 showing the upper region of the interior of the fluid cassette;

FIG. 8 is a perspective cross-sectional view of a portion of the fluid cassette showing a venting feature;

FIG. 9 is an exploded view of the fluid cassette of FIGS. 3 and 4;

FIG. 10 is a cross-sectional view of the fluid cassette showing the fluid pathway;

FIG. 11 is a cross-sectional view of a portion of the fluid pathway showing the mixing chamber; and

FIG. 12 is a cross-sectional view of a portion of the fluid cassette showing the arrangement of warming plates relative to the mixing chamber.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may control the flow of fluid into the body cavity and/or regulate body cavity pressure and/or the flow rate of fluid flow to the body cavity using an inflow and/or outflow pump of the fluid management system. The inflow pump may deliver fluid through inflow tubing of a fluid tubing set to the patient and/or the outflow pump may remove fluid through outflow tubing of a fluid tubing set from the patient. The fluid management system may include one or more sensors providing signals to the controller of the fluid management system to control the fluid flow.

In some instances in which the fluid management system is used in conjunction with an endoscope device such as, but not limited to, a LithoVue® Elite endoscope, the fluid management system may control the fluid flow using pressure and/or temperature data from the endoscope or other endoscopic device. Direct regulation of the intracavity pressure during a medical procedure using a pressure sensor on the endoscope may allow the fluid management system to safely control the fluid pressure with the body cavity.

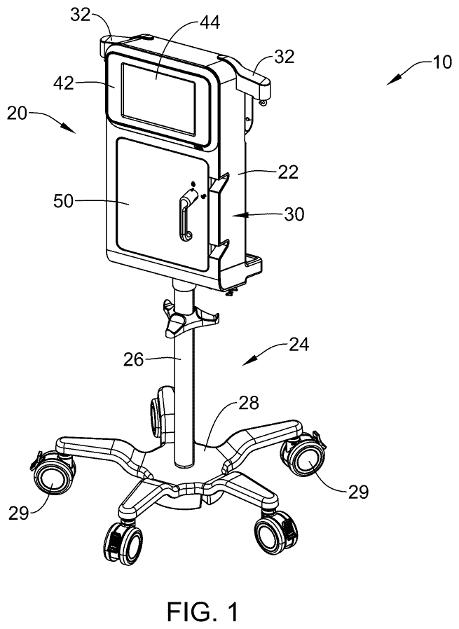

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the LithoVue® Elite endoscope, or other endoscope. In some instances, the endoscope may include a temperature sensor to provide intracavity temperature feedback to the fluid management system 10, a pressure sensor to provide intracavity pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The touch screen interface 42 allows the user to input/adjust various functions of the fluid management system 10 such as, for example flow rate, pressure, and/or temperature. The user may also configure parameters and alarms (such as, but not limited to, a max pressure alarm), information to be displayed, and the procedure mode. The touch screen interface 42 allows the user to add, change, and/or discontinue the use of various modular systems within the fluid management system 10. The touch screen interface 42 may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flow rate and/or fluid pressure display. In some embodiments, operating parameters may be adjusted by touching a corresponding portion of the touch screen interface 42. The touch screen interface 42 may also display visual alerts and/or audio alarms if parameters (e.g., flow rate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flow rate. Some illustrative display screens 44 and other user interface components are described in described in commonly assigned U.S. Patent Application Publication No. 2018/0361055, titled AUTOMATED FLUID MANAGEMENT SYSTEM, the entire disclosure of which is hereby incorporated by reference.

The touch screen interface 42 may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flow rate or the fluid pressure of fluid provided by the fluid management system 10 at any given time may be displayed on the display screen 44 to allow the operating room (OR) visibility for any changes. If the OR personnel notice a change in fluid flow rate or fluid pressure that is either too high or too low, the user may manually adjust the fluid flow rate or the fluid pressure back to a preferred level. The fluid management system 10 may also monitor and automatically adjust the fluid flow rate or the fluid pressure based on previously set parameters, as discussed herein.

An illustrative fluid management unit may include one or more fluid container supports, such as fluid supply source hanger(s) 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

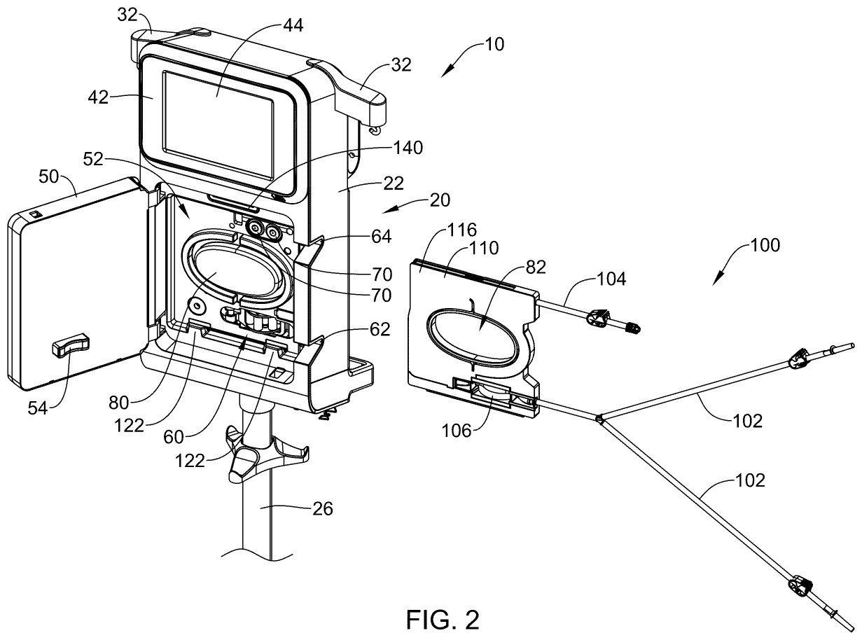

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flow rate or a target fluid pressure. As noted herein, the inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target fluid flow rate or target fluid pressure based on a set of system operating parameters. In some embodiments, the controller 30 may be configured to control the inflow pump 60 to maintain a desired fluid pressure at the treatment site or a desired flow rate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flow rate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flow rate, fluid pressure, and/or other parameters. Once the user has selected the manual mode on, for example, the touch screen interface 42, the user may the adjust fluid flow rate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the fluid inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the fluid inflow tubing 102, and the fluid outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flow rates required in the specific application of the fluid management system 10. Some illustrative fluid warming systems are described in described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

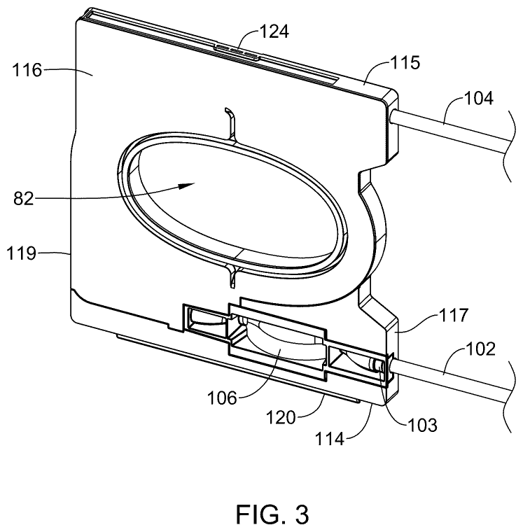

The console 20 may further include one or more additional sensors, such as a pressure sensor and/or a bubble sensor. For instance, the console 20 may include a pressure sensor 70, illustrated as a pair of pressure sensors, configured to monitor a system pressure of fluid exiting the cassette 110 and flowing through the outflow tubing 104 to a surgical site. The fluid cassette 110 may include a corresponding pressure sensor interface 72, such as a flexible membrane, (shown in FIG. 3) that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44.

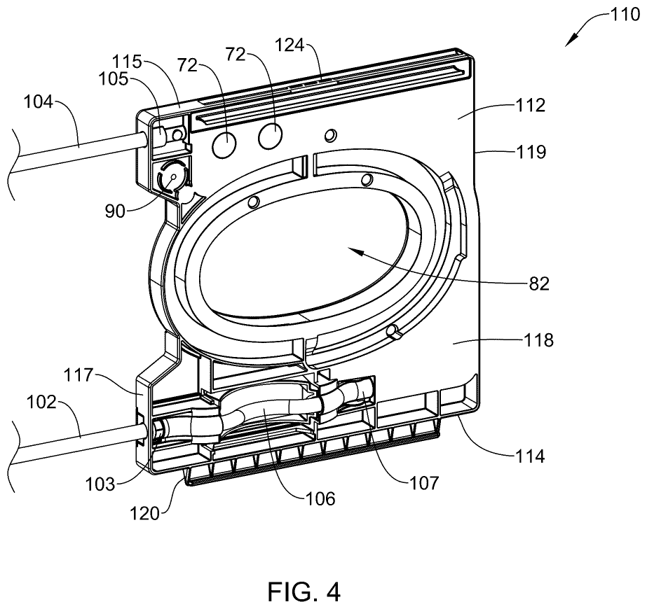

Additional features of the cassette 110 of the fluid tubing set 100 are illustrated in FIGS. 3 and 4. The fluid cassette 110 may include a housing 112 defining a fluid pathway through an interior of the housing 112. The fluid cassette 110 may include a front face 116 and a rear face 118 opposite the front face 116. The front face 116 is configured to face the door 50 of the console 20 when loaded in the receptacle 52, and the rear face 118 is configured to face a rear wall of the receptacle 52 of the console 20 when loaded in the receptacle 52. The fluid cassette 110 may also include an upper edge 115 and a lower edge 114 opposite the upper edge 115. The fluid cassette 110 may also include a first side edge 117 and a second side edge 119, opposite the first side edge 117. Both, the inflow tubing 102 and the outflow tubing 104 may extend from the first side edge 117. The housing 112 of the fluid cassette 110 may also include an opening 82, such as an oval opening, extending through the housing 112 from the front face 116 to the rear face 118. The opening 82 may extend a majority of the length of the housing 112 (i.e., a majority of the distance between the lateral edges of the housing 112) and/or a majority of the height of the housing 112 (i.e., a majority of the distance between the upper edge and the lower edge of the housing 112), in some instances. The opening 82 may be configured to receive an elevated portion of the rear wall of the receptacle 52, shown in FIG. 1 as the fluid warming system 80. The elevated portion of the rear wall of the receptacle 52 may be an oval shape sized to fit through the oval shaped opening 82 of the housing 112 of the fluid cassette 110 when the fluid cassette 110 is in its loaded position in the receptacle 52. In embodiments, in which the console 20 lacks a fluid warming system, the elevated portion of the rear wall of the receptacle 52 may still be present. Insertion of the elevated portion of the rear wall of the receptacle 52 through the opening 82 of the fluid cassette 110 may facilitate proper alignment of the fluid cassette 110 in the receptacle 52, for example.

In some embodiments, the fluid cassette 110 may include a fluid inlet port 103 and a fluid outlet port 105 located at a lateral side of the fluid cassette 110 accessible from the first side edge 117 of the fluid cassette 110. The fluid inlet port 103 may be coupled to the inflow tubing 102 and the fluid outlet port 105 may be coupled to the outflow tubing 104, with the inflow tubing 102 and the outflow tubing 104 extending laterally from the first side edge 117. The fluid inlet port 103 may be located below (e.g., closer to the lower edge 114) than the fluid outlet port 105. Thus, the inflow tubing 102 may extend laterally from the first side edge 117 at a location below (e.g., closer to the lower edge 114) than the location that the outflow tubing 104 extends from the first side edge 117. The cassette 110 may define an internal fluid pathway through an interior of the cassette housing 112 of the cassette 110 between the fluid inlet port 103 and the fluid outlet port 105. In embodiments of the fluid management system 10 including fluid warming capabilities, the internal fluid pathway may include the susceptor. The length of flexible pump tubing 106 of the cassette 110, configured to engage and be compressed by the rollers of the inflow pump 60, may extend from the fluid inlet port 103 to a connection 107 of the cassette 110 leading to the fluid pathway defined through the interior of the cassette 110. The flexible pump tubing 106 may be a discrete length of tubing separate from the inflow tubing 102 and the outflow tubing 104. In some instances, the flexible pump tubing 106 may extend through an arcuate pathway between the fluid inlet port 103 to the connection 107, such that the flexible pump tubing 106 follows the rotational path of the rollers of the inflow pump 60. The inlet port 103, the outlet port 105, and/or the connection 107 may be formed as a portion of the cassette housing 112, or formed separately and connected thereto.

The fluid cassette 110 may also include an air vent valve 90 configured to release air from the interior of the fluid cassette 110 to atmosphere. For example, the fluid cassette 110 may include an air vent including a hydrophobic membrane, allowing air, including bubbles entrained in the fluid, to pass through the hydrophobic membrane while preventing fluid within the fluid cassette 110 to pass therethrough. The air may then be vented to atmosphere through the air vent valve 90.

The fluid cassette 110 may also include one or more retention features configured to interact with the console 20 to retain the fluid cassette 110 in the receptacle 52 of the console 20. For example, the fluid cassette 110 may include a retention tab 120 extending from a lower edge of the housing 112 of the fluid cassette 110 and/or a retention tab 124 extending from an upper edge 115 of the housing 112 of the fluid cassette 110, configured to engage mating retention features of the console 20, as described in U.S. Provisional Application Ser. No. 63/597,481, entitled Fluid Management System With Disposable Fluid Cassette, filed on Nov. 9, 2023, the contents of which are hereby incorporated by reference in their entirety.

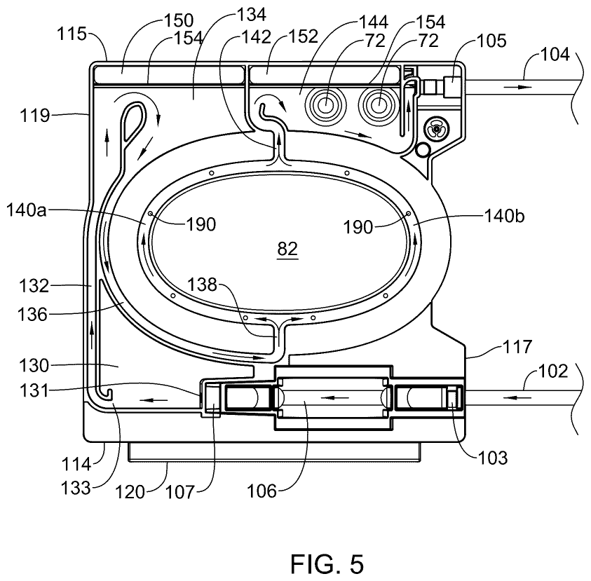

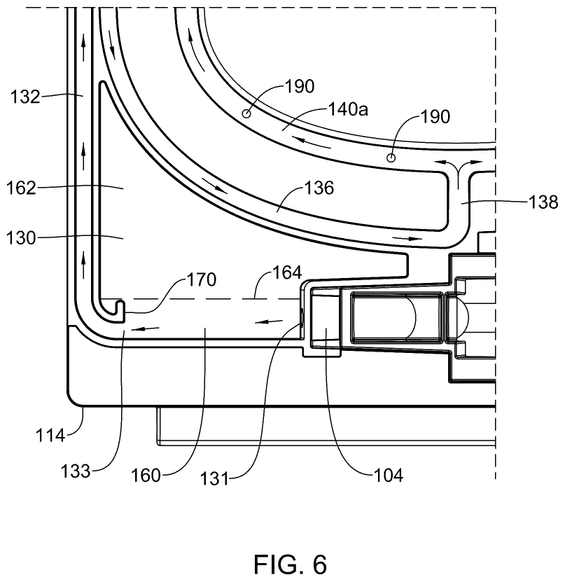

The internal flow pathway through the fluid cassette 110 is shown with arrows in the cross-sectional view of FIG. 5. Fluid flows into the interior of the fluid cassette 110 through the fluid inlet port 103 from the inflow tubing 102, and then passes through the pump tubing 106 as the pump tubing 106 is cyclically compressed by the rollers of the inflow pump 60. The fluid then flows into a fluid dampening chamber 130 configured to reduce pressure fluctuations of the pulsatile fluid flow exiting the pump tubing 106 created by the inflow pump 60, and thus smoothen the fluid flow as the fluid exits the fluid dampening chamber 130. The fluid dampening chamber 130 may include a single fluid inlet 131 and a single fluid outlet 133. The single fluid inlet 131 and the single fluid outlet 133 may be located on opposite sides of the fluid dampening chamber 130, such that fluid flows into the fluid dampening chamber 130 through the fluid inlet 131 and flows out of the fluid dampening chamber 130 through the fluid outlet 133. More details of the fluid dampening chamber 130 will be described herein, in regard to FIG. 6.

As fluid exits the fluid dampening chamber 130 through the fluid outlet 133 the fluid flows upward through the ascending fluid pathway 132. The ascending fluid pathway 132 interconnects the fluid dampening chamber 130 with a first air vent chamber 134. The fluid then exits the first air vent chamber 134 in a downward direction along a descending fluid pathway 136. As shown in FIG. 5, the descending fluid pathway 136 may be an arcuate pathway extending from an upper region above the oval opening 82 to a lower region below the oval opening 82.

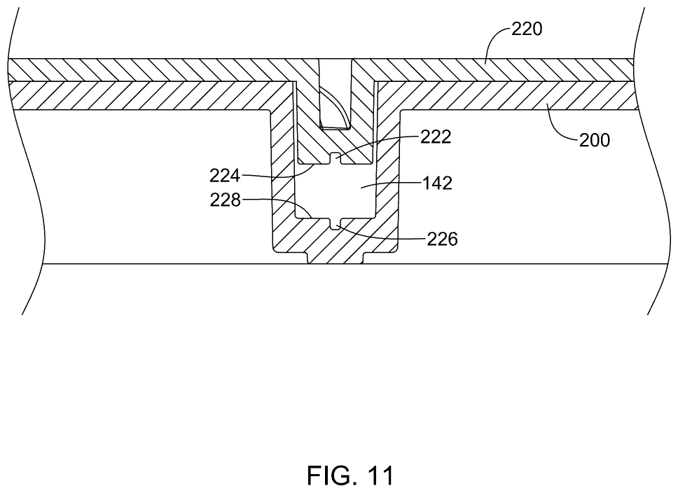

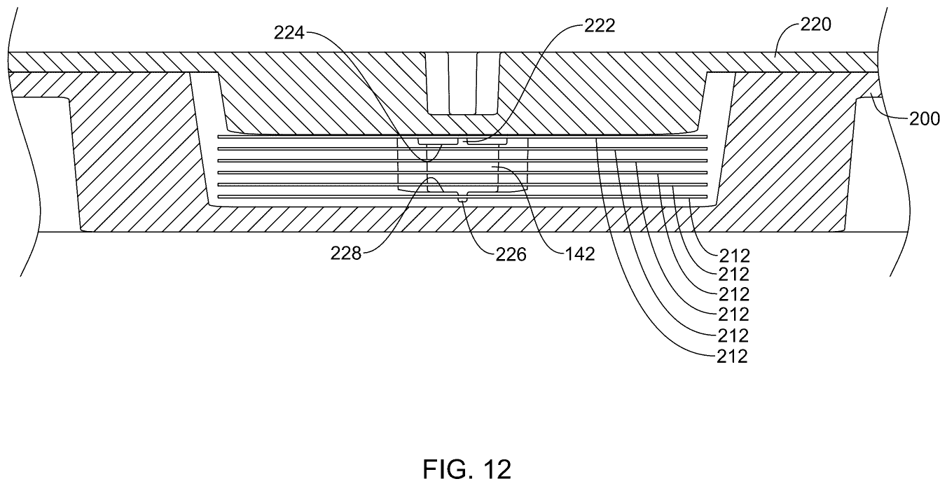

The fluid may then enter a bifurcated fluid pathway 140a/140b from the descending fluid pathway 136 as the fluid passes through a fluid warmer inlet channel 138 interconnecting the descending fluid pathway 136 and the bifurcated fluid pathway 140a/140b. The bifurcated fluid pathway 140a/140b includes a first fluid warming pathway 140a extending from the fluid warmer inlet channel 138 in a first direction and a second fluid warming pathway 140b extending from the fluid warmer inlet channel 138 in a second, generally opposite direction. The first fluid warming pathway 140a may extend around a first portion of the oval opening 82 on a first side of the oval opening 82 and the second fluid warming pathway 140b may extend around a second portion of the oval opening 82 on a second, opposite side of the oval opening 82. The bifurcated fluid pathway 140a/140b may then converge at a fluid mixing channel 142 located above the oval opening 82. Thus, the oval opening 82 may be located between the fluid mixing channel 142 and the fluid warmer inlet channel 138, such that the fluid mixing channel 142 is positioned above the oval opening 82 and the fluid warmer inlet channel 138 is positioned below the oval opening 82. More details of the fluid mixing channel 142 will be described herein, in regard to FIGS. 11 and 12.

It is noted that the bifurcated fluid pathway 140a/140b is described herein as defining first and second fluid warming pathways. In instances in which the fluid cassette and/or the console 20 include fluid warming capabilities, this is the region of the fluid pathway in which the fluid passing though the fluid cassette 110 may be warmed to an elevated temperature. However, in other instances in which the fluid cassette 110 and/or the console 20 does not include fluid warming capabilities, or in instances in which the fluid warming capabilities are disabled or deactivated (e.g., turned off), the bifurcated fluid pathway 140a/140b may still be described as including a first fluid warming pathway 140a and a second fluid warming pathway 140b. Thus, describing the pathways as “warming” pathways will be used throughout this disclosure regardless of whether fluid is actually being warmed in the pathways. The first fluid warming pathway 140a may alternatively be referred to as a first branch of the bifurcated fluid pathway and the second fluid warming pathway 140b may alternatively be referred to as a second branch of the bifurcated fluid pathway. Thus, the bifurcated fluid pathway may split into a first branch of the bifurcated fluid pathway and a second branch of the bifurcated fluid pathway as the bifurcated fluid pathway passes around the oval opening 82.

Returning to the flow pathway through the fluid cassette 110, fluid may flow upward from the fluid mixing channel 142 into a second air vent chamber 144. The fluid may then exit the second air vent chamber 144 to the outflow tubing 104 through the outlet port 105.

FIG. 6 is an enlarged view of the portion of the fluid cassette 110 including the fluid dampening chamber 130. During usage of the fluid cassette 110, a volume of fluid 160 may fill the lower portion of the fluid dampening chamber 130 while a volume of air 162 is trapped in the upper portion of the fluid dampening chamber 130. The fluid level 164 is the direct interface between the volume of fluid 160 and the volume of air 162. The fluid dampening chamber 130 may be designed to substantially smoothen the pulsatile fluid flow from the inflow pump 60 for fluid flows up to 800 ml/min, in some instances. For example, it has been found that sizing the fluid dampening chamber 130 such that the volume of air 162 is at least 38 ml substantially smoothens the pulsatile fluid prior to exiting the fluid dampening chamber 130. Accordingly, the fluid dampening chamber 130 may be sized to provide a volume of air 162 of 38 ml or more, or 40 ml or more, in some instances. For instance, the fluid dampening chamber may be sized to provide a volume of air 162 of 38 ml to 42 ml, during use.

Furthermore, as noted above, the fluid dampening chamber 130 may include a single fluid inlet 131 and a single fluid outlet 133 located on opposite sides of the fluid dampening chamber 130, such that fluid flows into the fluid dampening chamber 130 through the fluid inlet 131 and flows out of the fluid dampening chamber 130 through the fluid outlet 133. The fluid inlet 131 and the fluid outlet 133 may be positioned near a base of the fluid dampening chamber 130. The fluid dampening chamber 130 may be configured such that the upper extent of the fluid outlet 133 is lower (i.e., closer to the lower edge 114 of the fluid cassette 110) than the upper extent of the fluid inlet 131. This ensures that the fluid level 164 is above the upper extent of the fluid outlet 133 such that air from the volume of air 162 is not pulled out of the fluid dampening chamber 130 into fluid exiting the fluid dampening chamber 130 though the fluid outlet 133 into the ascending fluid pathway 132, which could otherwise occur at high flow rates. In some instances, the fluid outlet 133 may include a lip 170 extending upward from the upper extent of the opening of the fluid outlet 133 into the fluid dampening chamber 130. The lip 170 may have any desired height. In some instances, the height of the lip 170 may be sized such that the fluid level 164 is above the upper extent of the lip 170. In other instances, the fluid level 164 may impinge the lip 170.

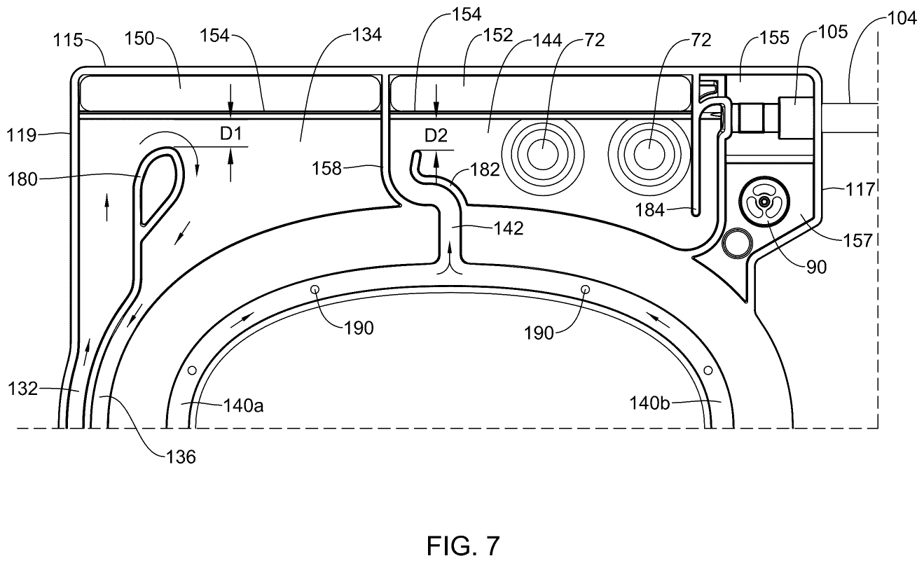

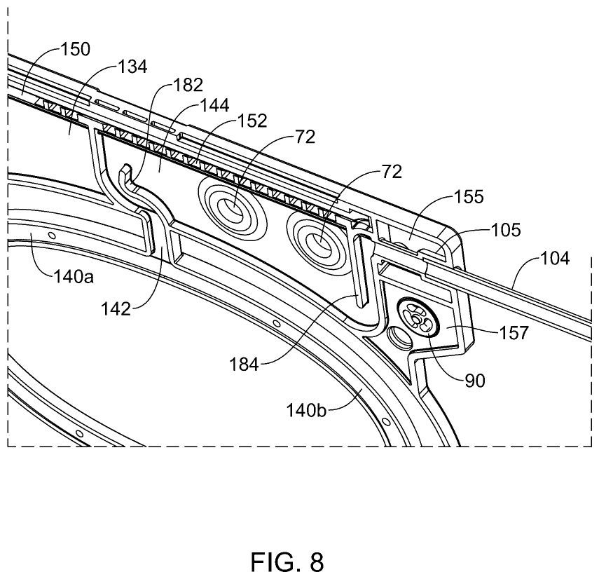

FIG. 7 is an enlarged cross-sectional view of the upper region of the fluid cassette 110, and FIG. 8 is a perspective cross-sectional view of the upper region of the fluid cassette to further describe the air venting features of the fluid cassette 110. As discussed above, the fluid pathway through the interior of the fluid cassette 110 includes a first air vent chamber 134 and a second air vent chamber 144. The bifurcated fluid pathway 140a/140b may be located between the first air vent chamber 134 and the second air vent chamber 144 along the fluid pathway. Thus, the first air vent chamber 134 may be configured to remove any entrained air bubbles in the fluid exiting the fluid dampening chamber 130 and the second air vent chamber 144 may be configured to remove any entrained air bubbles in the fluid exiting the first and second fluid warming pathways 140a/140b of the bifurcated fluid pathway.

A hydrophobic membrane 154 may be provided with each of the first and second air vent chambers 134/144. For example, a first hydrophobic membrane 154 may be positioned at an interface between the first air vent chamber 134 and a first air chamber 150. The interface including the first hydrophobic membrane 154 may be located along an upper extent of the first air vent chamber 134 such that air may pass through the first hydrophobic membrane 154 into the first air chamber 150 while the fluid in the first air vent chamber 134 is prevented from passing through the first hydrophobic membrane 154. A second hydrophobic membrane 154 may be positioned at an interface between the second air vent chamber 144 and a second air chamber 152. The interface including the second hydrophobic membrane 154 may be located along an upper extent of the second air vent chamber 144 such that air may pass through the second hydrophobic membrane 154 into the second air chamber 152 while the fluid in the second air vent chamber 144 is prevented from passing through the second hydrophobic membrane 154.

A wall 158 may be located between and separating the first air vent chamber 134 from the second air vent chamber 144 such that the first air vent chamber 134 is not directly fluidly connected to the second air vent chamber 144, but rather the first air vent chamber 134 is in fluid communication with the second air vent chamber 144 only via the bifurcated fluid pathway 140a, 140b.

The first air vent chamber 134 may include a deflector 180, configured as an interior wall within the first air vent chamber 134, that helps direct fluid flowing upward from the ascending fluid flow pathway 132 toward the upper extent of the first air vent chamber 134 and the first hydrophobic membrane 154. An upper extent of the deflector 180 may be located closer to the upper extent of the first air vent chamber 134 than to a lower extent of the first air vent chamber 134. In some instances, the upper extent of the deflector 180 may be located within 0.8 inches or less, within 0.7 inches or less, within 0.6 inches or less, within 0.5 inches or less, or within 0.4 inches or less of the upper extent of the first air vent chamber 134 and the first hydrophobic membrane 154. Accordingly, the distance D₁ between the upper extent of the deflector 180 and the first hydrophobic membrane 154 may be in the range of about 0.3 inches to about 0.7 inches, in the range of about 0.3 inches to about 0.5 inches, or in the range of about 0.35 inches to about 0.4 inches, for example.

It is also noted that the first air vent chamber 134 may be configured with the fluid inlet into the first air vent chamber 134 from the ascending fluid pathway 132 above (i.e., closer to the upper edge 115 of the fluid cassette 110) the fluid outlet from the first air vent chamber 134 into the descending fluid pathway 136. Such as configuration may facilitate fluid flow adjacent to the first hydrophobic membrane 154 prior to fluid exiting the first air vent chamber 134.

The second air vent chamber 144 may include a deflector 182, configured as an interior wall within the second air vent chamber 144, that helps direct fluid flowing upward from the fluid mixing channel 142 toward the upper extent of the second air vent chamber 144 and the second hydrophobic membrane 154. An upper extent of the deflector 182 may be located closer to the upper extent of the second air vent chamber 144 than to a lower extent of the second air vent chamber 144. In some instances, the upper extent of the deflector 182 may be located within 0.8 inches or less, within 0.7 inches or less, within 0.6 inches or less, within 0.5 inches or less, or within 0.4 inches or less of the upper extent of the second air vent chamber 144 and the second hydrophobic membrane 154. Accordingly, the distance D₂ between the upper extent of the deflector 182 and the second hydrophobic membrane 154 may be in the range of 0.3 inches to about 0.7 inches, in the range of about 0.3 inches to about 0.5 inches, in the range of about 0.4 inches to about 0.5 inches, or in the range of about 0.4 inches to about 0.45 inches, for example.

It is also noted that the second air vent chamber 144 may be configured with the fluid inlet into the second air vent chamber 144 from the fluid mixing channel 142 above (i.e., closer to the upper edge 115 of the fluid cassette 110) the fluid outlet from the second air vent chamber 144 into the fluid outlet port 105. For example, the second air vent chamber 144 may also include a deflector 184 defining a fluid outlet from the second air vent chamber 144 to the outflow port 105. The deflector 184 may position the fluid inlet into the second air vent chamber 144 higher (i.e., closer to the upper edge 115 of the fluid cassette 110) than the fluid outlet from the second air vent chamber 144. Such as configuration may facilitate fluid flow adjacent to the second hydrophobic membrane 154 prior to fluid exiting the second air vent chamber 144.

The pressure sensor interface(s) 72 may be located in a wall of the second air vent chamber 144 such that the pressure sensor(s) 70, discussed above, can monitor the fluid pressure of the fluid within the fluid cassette 110 just prior to the fluid exiting the fluid cassette 110. For instance the pressure sensor interface(s) 72 may be a flexible membrane that flexes against the pressure sensor(s) on an exterior thereof, as the pressure of the fluid within the second air vent chamber 144 impinges upon the interior surface of the flexible membrane. The fluid pressure monitored may be considered a system pressure, for example, which may be utilized by the controller 30 to adjust the inflow pump 60 to maintain a desired pressure during a medical procedure.

The first air chamber 150 may be interconnected with the second air chamber 152, and the combination for the first air chamber 150 and the second air chamber 152 may vent air to atmosphere through the air vent valve 90. For example, as shown in FIG. 8, the air in the first air chamber 150 and the second air chamber 152 may pass into an air chamber 155 defined as a cavity of the housing 112 of the fluid cassette 110 surrounding the outlet port 105. In turn, the air chamber 155 may be connected to the air chamber 157 defined as a cavity of the housing 112 of the fluid cassette 110 in direct communication with the air vent valve 90. Accordingly, air may be vented from the first air chamber 150 and/or the second air chamber 152 through the air chambers 155, 157 to exit the fluid cassette 110 via the air vent valve 90.

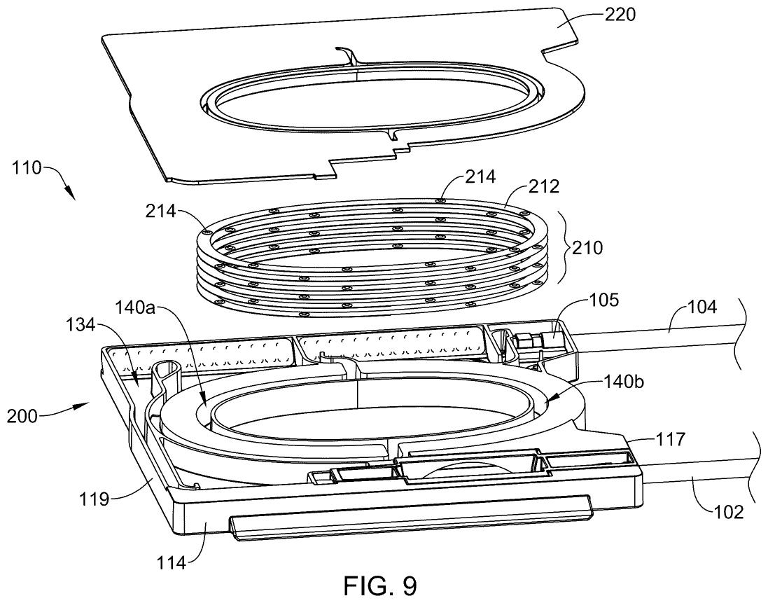

FIG. 9 is an exploded view of the fluid cassette 110. The housing 112 of the fluid cassette 110 may be formed of multiple components, that when assembled together form the fluid cassette 110. For example, the housing 112 may include a base 200 and a cover 220. The base 200 may include a plurality of interior walls defining the fluid pathway through the interior of the fluid cassette 110. The cover 220 may extend across the interior walls. In instances in which the fluid cassette 110 includes fluid warming capabilities, the fluid cassette 110 may include a stack of heating plates 210. The stack of heating plates 210 may be disposed within the bifurcated fluid pathway 140a/140b such that fluid passes directly across the stack of heating plates 210 as the fluid passes through the bifurcated fluid pathway 140a/140b to transfer heat from the stack of heating plates 210 to the fluid. For example, the stack of heating plates 210 may heat the fluid through an induction heating process.

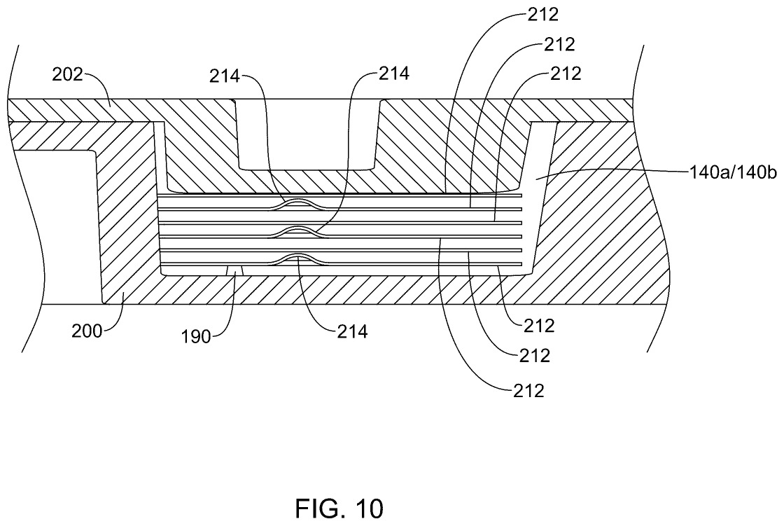

The stack of heating plates 210 may include a plurality of annular plates 212 stacked one on top of the other. The annular plates 212, which may be ring-shaped, may be formed of a metal material, such as stainless steel, for example. In some instances, the annular plates 212 may be circular or oval plates, with a central opening. Each of the plates 212 may include a flat upper surface and a flat lower surface, opposite the upper surface. The annular plates 212 may be stacked on top of each other, such that each plate 212 is spaced apart from adjacent plates to allow fluid to flow therebetween. In other words, the annular plates 212 may be configured such that there is a gap between the facing surfaces of the plurality of plates 212 (i.e., the upper surface of one plate 212 and the lower surface of a second, adjacent plate 212) to allow fluid to flow between the adjacent plates 212. For instance, the annular plates 212 may include, spacers, such as dimples 214 extending from one of the flat surfaces of the annular plates 212 (i.e., the upper and/or lower flat surfaces). The dimples 214, or other type of spacers, may be intermittently arranged around the perimeter of the annular plates 212. Turning to FIG. 10, the raised dimples 214 may contact an upper/lower surface of an adjacent plate 212, retaining a gap between the adjacent plates 212 for fluid flow. As shown in FIG. 10, the annular plates 212 may be arranged in the bifurcated fluid pathway 140a/140b defined between a surface of the cover 220 and the base 200 of the housing 112 of the fluid cassette 110. In other instances, one or more of the plates 212 may include a different raised structure to space apart the adjacent plates 212. For example, the spacers may be raised ridges, tabs, or other structures configured to contact an adjacent plate 212 to maintain a gap therebetween. In other instances, the spacers may be separate components placed between adjacent plates 212 and/or a portion of the housing 112 maintaining the adjacent plates 212 in a spaced apart configuration with a gap for fluid flow therebetween.

Furthermore, as shown in FIG. 10, the interior surface of the base 200 and/or the cover 220 may include one or more spacers extending therefrom to contact a plate 212 and space the plate 212 apart from the interior surface of the base 200 and/or the cover 220. For instance, the spacers may be projections 190 extending into the bifurcated fluid pathway 140a/140b to space the uppermost plate 212 of the stack of heating plates 210 from the interior surface of the cover 220 and/or the lowermost plate 212 of the stack of heating plates 210 from the interior surface of the base 200 of the housing 112.

As discussed above, as fluid exits the bifurcated fluid pathway 140a/140b the fluid enters the fluid mixing channel 142. FIG. 11 is a cross-sectional view taken through the fluid mixing channel 142. In embodiments in which the fluid cassette 110 has fluid warming capabilities and is used to warm fluid with the stack of heating plates 210, the fluid may not be uniformly heated or warmed. For example, fluid toward the middle of the fluid flow may be heated to a higher temperature than fluid toward the upper extent (closer to the uppermost plate 212) of the fluid flow and/or fluid toward the lower extent (closer to the lowermost plate 212) of the fluid flow. This phenomena may be attributed to the fluid flowing across the middle plates 212 being in contact with more surface area of the plurality of plates 212 than fluid flowing across or in contact with the uppermost and/or lowermost plates 212. Accordingly, it may be desirable to mix the fluid exiting the fluid warming pathways (i.e., the first fluid warming pathway 140a and the second fluid warming pathway 140b to more evenly distribute the elevated temperature of the fluid.

The fluid mixing channel 142 may be configured to generate turbulence in the fluid flow, or otherwise mix the fluid exiting the bifurcated fluid warming pathways 140a/140b. For example, the interior surface 224 of the cover 220 and/or the interior surface 228 of the base 200 defining the fluid mixing channel 142 may include a recess 222/226 through which some of the fluid in the fluid mixing channel 142 passes through. In other instances, the fluid mixing channel 142 may have a different configuration. For example, the fluid mixing channel 142 may have one or more helical ridges, one or more corrugations, one or more baffles, or other structures to generate turbulence or mixing of the heated fluid exiting the bifurcated fluid warming pathways 140a/140b.

FIG. 12 is a cross-sectional view through the fluid cassette 110 showing the stack of heating plates 210 positioned across the entrance to the fluid mixing channel 142. As can be seen, the uppermost plate 212 of the stack of heating plates 210 may extend across the recess 222 defined in the interior surface 224 of the cover 220 of the housing 112 of the fluid cassette 110 and/or the lowermost plate 212 of the stack of heating plates 210 may extend across the recess 226 defined in the interior surface 228 of the base 200 of the housing 112 of the fluid cassette 110. The inclusion of the recesses 222/226, or other fluid mixing structure, has been found to more evenly distribute the elevated temperature of the fluid exiting the fluid mixing channel 142.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid cassette for a fluid management system, comprising:
a housing defining a fluid pathway therethrough;
an inflow tubing extending from the housing;
an outflow tubing extending from the housing;
wherein the fluid pathway includes a fluid dampening chamber having a fluid inlet configured for fluid ingress into the fluid dampening chamber and a fluid outlet configured for fluid egress from the fluid dampening chamber;
wherein an upper extent of the fluid outlet is lower than an upper extent of the fluid inlet.

2. The fluid cassette of claim 1, wherein the fluid inlet and the fluid outlet are located on opposite sides of the fluid dampening chamber.

3. The fluid cassette of claim 1, wherein the fluid dampening chamber is configured such that a fluid level between a volume of air and a volume of fluid within the fluid dampening chamber is higher than the upper extent of the fluid outlet.

4. The fluid cassette of claim 3, wherein the volume of air is at least 38 milliliters.

5. The fluid cassette of claim 1, wherein the fluid pathway includes a first air vent chamber and an ascending fluid channel interconnecting the fluid dampening chamber and the air vent chamber.

6. The fluid cassette of claim 5, wherein the fluid pathway includes a bifurcated fluid pathway including a first branch of the bifurcated fluid pathway and a second branch of the bifurcated fluid pathway, and a descending fluid channel interconnecting the first air vent chamber with the bifurcated fluid pathway.

7. The fluid cassette of claim 6, wherein the fluid pathway includes a second air vent chamber and a mixing fluid channel interconnecting the bifurcated fluid pathway with the second air vent chamber.

8. The fluid cassette of claim 7, further comprising an interior wall separating the first air vent chamber from the second air vent chamber.

9. A fluid cassette for a fluid management system, comprising:
a housing defining a fluid pathway therethrough;
an inflow tubing extending from the housing;
an outflow tubing extending from the housing;
wherein the fluid pathway includes a first air vent chamber and a second air vent chamber;
wherein the first air vent chamber and the second air vent chamber are fluidly connected by a fluid pathway therebetween.

10. The fluid cassette of claim 9, wherein the fluid pathway interconnecting the first air vent chamber and the second air vent chamber is a bifurcated fluid pathway including a first branch of the bifurcated fluid pathway and a second branch of the bifurcated fluid pathway.

11. The fluid cassette of claim 9, further comprising:
a first hydrophobic membrane positioned at an interface between the first air vent chamber and a first air chamber, wherein air may pass through the first hydrophobic membrane into the first air chamber while fluid in the first air vent chamber is prevented from passing through the first hydrophobic membrane, and
a second hydrophobic membrane positioned at an interface between the first air vent chamber and a second air chamber, wherein air may pass through the second hydrophobic membrane into the second air chamber while fluid in the second air vent chamber is prevented from passing through the second hydrophobic membrane;
wherein the first air chamber is interconnected with the second air chamber.

12. The fluid cassette of claim 11, wherein the first air chamber and the second air chamber are configured to vent air to atmosphere through an air vent valve.

13. The fluid cassette of claim 9, wherein the first air vent chamber includes a deflector configured as an interior wall, wherein an upper extent of the deflector in the first air vent chamber is located closer to an upper extent of the first air vent chamber than to a lower extent of the first air vent chamber.

14. The fluid cassette of claim 9, wherein the second air vent chamber includes a deflector configured as an interior wall, wherein an upper extent of the deflector of the second air vent chamber is located closer to an upper extent of the second air vent chamber than to a lower extent of the second air vent chamber.

15. A fluid cassette for a fluid management system, comprising:
a housing defining a fluid pathway therethrough;
an inflow tubing extending from the housing;
an outflow tubing extending from the housing;
wherein the fluid pathway includes a bifurcated fluid pathway including a first branch of the bifurcated fluid pathway and a second branch of the bifurcated fluid pathway.

16. The fluid cassette of claim 15, wherein fluid exiting the first and second branches of the bifurcated fluid pathway enters a fluid mixing channel configured to generate a turbulent flow to mix the fluid exiting the first and second branches of the bifurcated fluid pathway.

17. The fluid cassette of claim 15, wherein the first branch of the bifurcated fluid pathway is a first fluid warming pathway and the second branch of the bifurcated fluid pathway is a second fluid warming pathway.

18. The fluid cassette of claim 17, further comprising a stack of heating plates arranged in the first and second fluid warming pathways.

19. The fluid cassette of claim 18, wherein the stack of heating plates includes a plurality of spaced apart annular plates configured to permit fluid to pass therebetween.

20. The fluid cassette of claim 19, wherein each of the annular plates includes one or more dimples extending from a flat surface of the annular plate, wherein the one or more dimples of one of the annular plates is configured to contact a second one of the annular plates to space the first annular plate away from the second annular plate.
